(19) United States
(12) Patent Application Publication
(10) Pub. No.: US 2006/0276870 A1
(43) Pub. Date: Dec. 7, 2006

(54) Osseus stimulating electrodes

(76) Inventor: William J. McGinnis, Cincinnati, OH (US)

Correspondence Address: LADAS & PARRY LLP, 224 SOUTH MICHIGAN AVENUE, SUITE 1600, CHICAGO, IL, 60604, US

(21) Appl. No.: 11/373,361

(22) Filed: Mar. 10, 2006

Related U.S. Application Data
(63) Continuation-in-part of application No. 11/292,861, filed on Dec. 2, 2005.
Application No. 11/292,861 is a continuation-in-part of application No. 11/144,214, filed on Jun. 3, 2005.

(51) Int. Cl. A61N 1/18

(52) U.S. Cl. 607/145; 607/48

(57) ABSTRACT

This invention provides surgical instruments for eliciting motor evoked potentials intraoperatively during surgical procedures such as anterior cervical discectomy and fusions by providing a stimulus directly to an osseus structure such as a vertebral body.

### CROSS REFERENCE TO RELATED APPLICATIONS

This application is a continuation-in-part of application Ser. No. 11/292,861 filed Dec. 2 2005, and Ser. No. 11/144,214filed Jun. 3 2005, which are incorporated by reference herein in their entirety.

### TECHNICAL FIELD

This invention relates to surgical instruments for eliciting motor evoked potentials, and more particularly, to a surgical instrument for providing a stimulus directly to an anterior cervical vertebral body.

### BACKGROUND

Avoiding paralysis is a major intraoperative concern, and the most functionally significant impairments following spinal surgery are probably related to motor deficits. Motor evoked potentials (MEP) assess the motor systems from the motor cortex to the anterior horn cell and then by way of the peripheral nerves to the muscle, and monitoring these pathways should theoretically permit better detection of intraoperative motor function loss. Motor evoked potentials are recorded from either peripheral muscles (myogenic motor evoked potentials, MMEPS) or from peripheral nerves (neurogenic motor evoked potentials, NMEPs) and can be generated by applying either electrical or magnetic stimulation transcranially to the motor cortex or to the spinal cord.

Transcranial electrical stimulation of the motor cortex is a reliable method of eliciting motor evoked potentials, achieved by delivering brief high-voltage pulses through scalp electrodes. It is recorded from the spinal cord, the epidural space, the peripheral nerves and the musculature using conventional electromyographic and evoked potential averaging techniques. Transcranial magnetic stimulation is produced by placing a magnetic coil over the motor cortex. However, transcranial electrode placement and stimulation is time intensive and technically demanding intraoperatively. With transcranial magnetic or electrical stimulation, there is also concern that repetitive cortical stimulation can induce epileptic activity, neural damage and cognitive or memory dysfunction. Other situations of concern are patients with cardiac pacemakers and central venous or pulmonary artery catheterization. Furthermore, the clinical utility of transcranial motor evoked potentials is severely limited due to the large attenuation of evoked responses caused by most anesthetic agents.

Direct spinal cord stimulation has been used to assess the motor pathways with recordings obtained from the lower extremities using either surface or intramuscular electromyography to assess the motor pathways.

To date however, only posterior elements of the spinal cord such as such as the lamina have been stimulated, and it is believed that stimulating the posterior elements of the spinal cord may fail to achieve stimulation of the motor pathway, and thus may fail to produce an evoked potential.

Thus, there remains a need for a way monitoring the corticospinal tract's functional integrity intraoperatively.

It has been surprisingly found that a motor evoked potential is reliably elicited by providing a stimulus directly to the corticospinal tract (motor pathway), via stimulation of the anterior cervical vertebral bodies. The electrical resistance of the inherent positive charge of the bone is overcome and propagation of the electrical current follows the path of least resistance to the anterior surface of the spinal cord. The changes in electrical charge generated by the current, elicits conductive changes at the level of the corticospinal tract, thereby activating the motor pathways. The path of the stimulus when applied to the anterior cervical vertebral bodies is: vertebral body (anterior cervical spine)→anterior spinal cord→corticospinal tract (motor pathways).

It has also been surprisingly found that anterior cord stimulated motor evoked potentials are resistant to attenuation induced by general anesthesia. Furthermore, a device for providing stimulation directly to the spinal cord within a bony body or nerve tissue has so far eluded the industry.

A stimulating instrument and technique to reliably elicit motor evoked potentials intraoperatively would be of importance.

### SUMMARY OF THE INVENTION

The present invention addresses the problems outlined above by providing a stimulating surgical instruments for eliciting motor evoked potentials, comprising an osseus structure probe having a probe distal end, the probe distal end being open, and a stimulating wire having wire proximal and distal ends, the stimulating wire extending through the probe and being exposed in the open distal end, such that when the probe distal end is embedded in an osseus structure of a subject, and when the stimulating wire proximal end is in contact with a stimulus source, the exposed wire distal end is capable of delivering an electrical stimulus to the surrounding osseus structure.

In another view of the invention, the invention is also directed to systems and methods are provided for eliciting motor evoked potentials intraoperatively. A system is provided comprising one or more of the instrument as described, and at least a processor. In a preferred embodiment the system comprises software for controlled delivery of stimulus, data feedback and real-time monitoring.

The invention is further directed to a method for eliciting an evoked potential in a subject during a surgical procedure being performed on the subject by delivering a stimulus to the osseus structure of the subject. A surgical instrument comprising an osseus structure probe and a stimulating wire extending through the probe to, and being exposed at its distal end at, the open probe distal end, is connected to a subject's osseus structure. The proximal end of the stimulating wire is put in connection with a stimulus source and a stimulus is delivered to the osseus structure via the exposed wire distal end. Then an evoked potential elicited from the subject as a result of the delivered stimulus is measured.

It should be appreciated that the present invention can be implemented and utilized in numerous ways, including without limitation a device, an apparatus, a system, and a method for applications now known and later developed. These and other unique features of the system disclosed herein will become more readily apparent from the following description and the accompanying drawings.

### DESCRIPTION OF DRAWINGS

FIG. 1 is a perspective view of an osseus structure stimulating surgical instrument.

FIG. 2 shows a front or side elevation of an osseus structure stimulating surgical instrument.

FIG. 3 shows an alternative embodiment of an osseus stimulating instrument embedded in bone or an osseus structure.

FIG. 4 shows an enlarged view of the distal end of an osseus stimulating instrument.

FIG. 5 shows an alternative embodiment of an osseus stimulating instrument.

FIG. 6 illustrates a system comprising an osseus structure stimulating surgical instrument.

Like reference symbols in the various drawings indicate like elements.

### DETAILED DESCRIPTION

The invention provides surgical instruments for directly stimulating osseus structures, providing a stimulus to the anterior cervical spine via the anterior vertebral bodies and reliably eliciting motor evoked potentials intraoperatively.

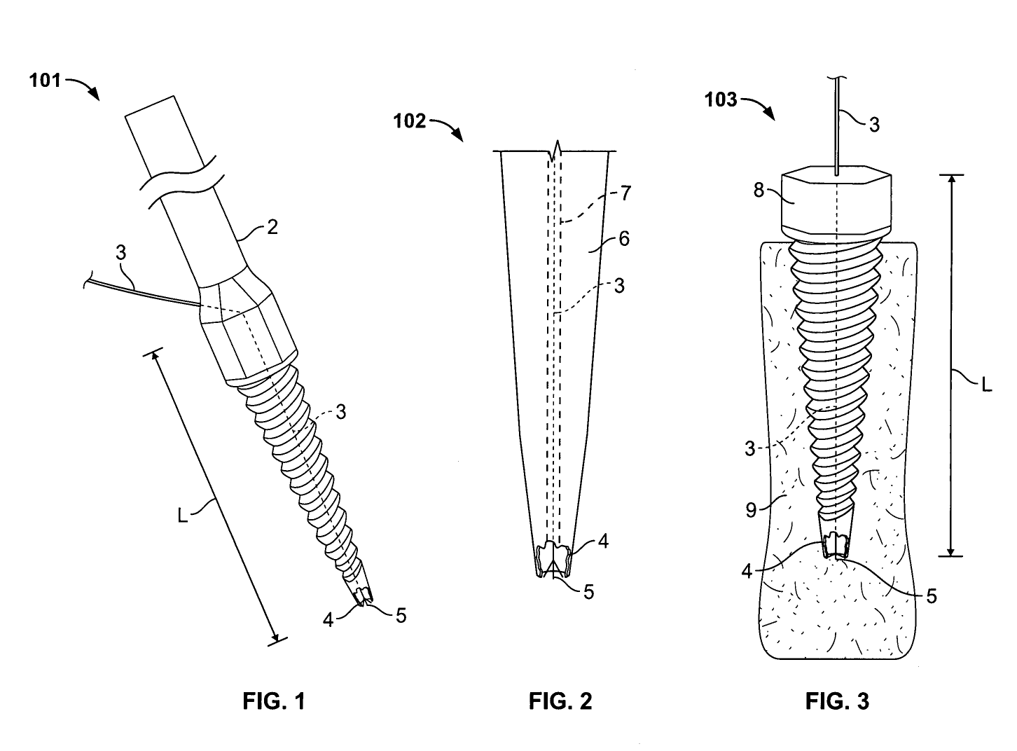

The FIGS. 1-6 show various embodiments of the stimulating surgical instrument. FIG. 1 shows an embodiment of stimulating surgical instrument (101) such as a vertebral distractor, in which probe (2) illustratively comprises a screw lower portion for facilitating embedding in a target osseus structure. The screws on the exterior of the lower portion of the probe (2) allow the probe to be screwed into bone.

In FIG. 2, probe (2) has in-housed stimulating wire (3) extending down probe length (L) through probe (2) down to an open distal probe end (4) where distal wire end (5) is exposed to allow a stimulus, or an electrical stimulating signal, to be delivered to the osseus structure surrounding the distal probe end when the instrument is embedded in the osseus structure. The proximal end of the wire can optionally enter the instrument at the side or top of the instrument as is illustratively shown respectively in FIGS. 1 and 3.

In a preferred embodiment, the probe is made of surgical stainless steel, in which case the stimulating wire requires insulation along the length of the wire extending through an internal tube in the probe to allow electrical stimulating signals to be delivered to the distal end. FIG. 2 shows an embodiment (102) of the instrument in which probe distal portion (6) comprises a tubular portion (7) in-housing stimulating wire (3). Where the probe is made of a conducting material, tube (7) needs only be large enough to accommodate the outside diameter of the stimulating wire. In an alternative embodiment, the probe shown in FIG. 1 could also be made of a material other than stainless steel, such as ceramic, quartz, plastics or glass, and in such embodiments, the stimulating wire need not be insulated along the length of the probe.

FIG. 3 shows a screw instrument embodiment (103) in which stimulating wire (3) enters probe (8) at the top or proximal end of the probe and extends along length L down through the probe to open probe distal end (4) where the stimulating wire (3) is exposed to surrounding osseus structure (9) in which probe (8) is embedded. In the embodiment of FIG. 3, the exterior of the probe is threaded to enable the probe to be screwed into bone.

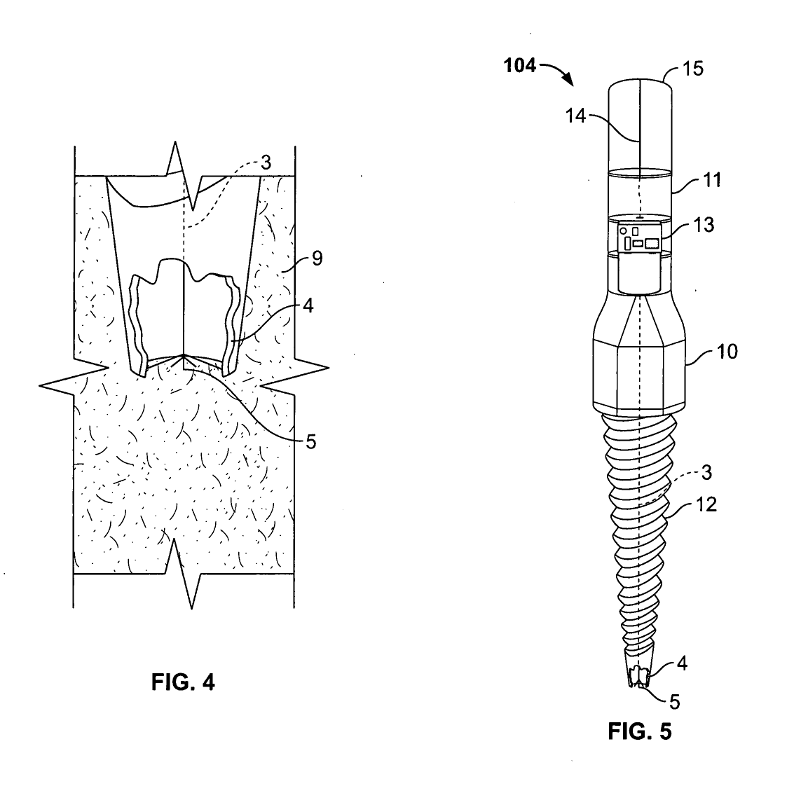

FIG. 4 shows an enlargement of a probe distal end embedded in osseus tissue (9) in which stimulating wire (3) extends to open probe distal end (4) where distal wire end (5) is exposed to the surrounding tissue.

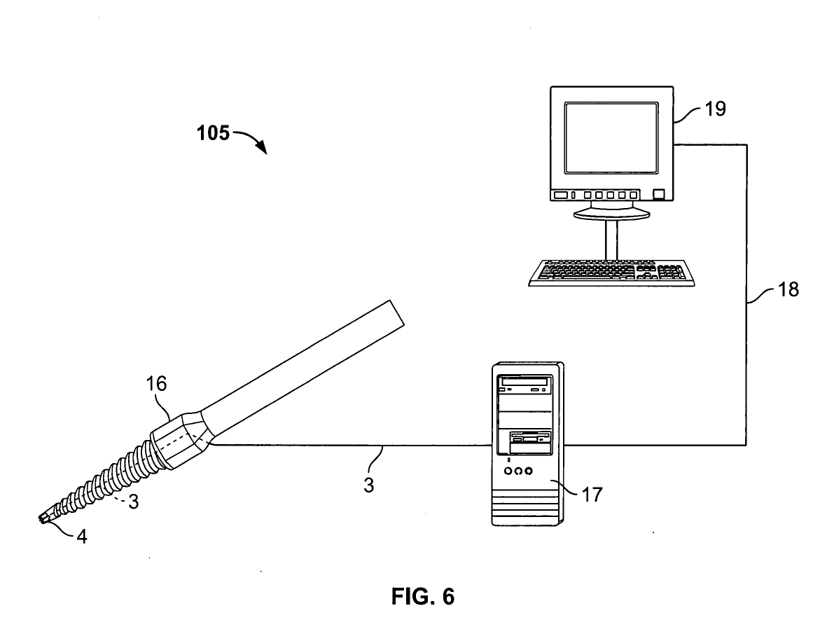

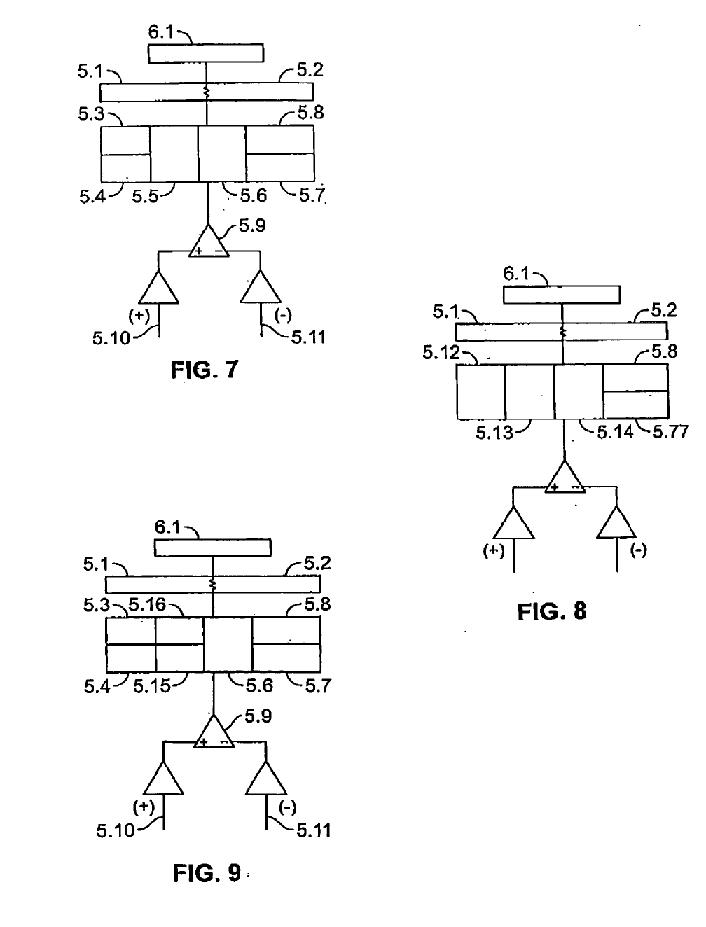

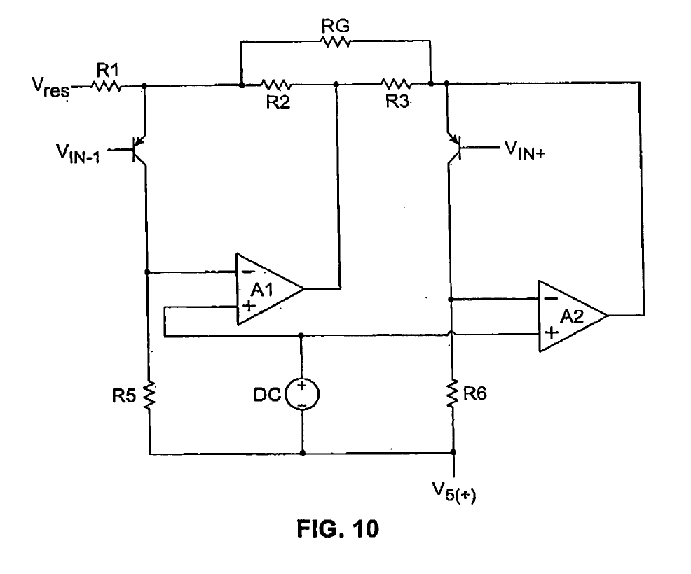

FIG. 5 shows instrument embodiment (104) in which probe (10) comprises a top portion (11) and a screw end (12), the screw end having an open distal end (4) to which stimulating wire (3) extends and is exposed at distal wire end (5). Probe top portion (11) comprises biosensor (13) in contact with stimulating wire (3) and also in connection to light receiver (14). Light receiver (14) has transparent dome (15) via which it is able to receive power wirelessly from a remote light source as described more fully below.

FIG. 6 shows one embodiment (105) of the inventive system comprising probe (16) with in-housed stimulating wire (3) exposed its distal end at open probe distal end (4) and connected directly to stimulus source and processor (17) which is hard-wired via (18) to user display and real-time monitoring system (19).

The invention provides therefore stimulating surgical instruments that can reliably elicit motor evoked potentials. The preponderance of anterior discectomies and fusions at level specific sites employ the use of devices such as a vertebral body spreader, or vertebral body post distractor (vertebral body post), to expose the disc space. Other commonly used devices during spinal surgeries are pins, posts and needles that are capable of being embedded in a subject's osseus structure such as a vertebral body.

The herein described stimulating surgical instruments based on such devices for providing an electrical stimulus to an osseus structure such as a vertebral body, comprising a probe suitable for osseus structures such as a vertebral body having an in-housed stimulating wire for providing a stimulus to the osseus structure. The stimulating wire extends down through the probe to an opening in the probe distal end where the wire distal end is exposed. When the probe is embedded in bone or osseus structure, and when the wire proximal end is put in contact with a stimulus source, the exposed wire distal end is capable of delivering an electrical stimulus to the surrounding bone or osseus structure. The stimulating wire may be exposed up to 2 mm from its distal end.

By distal end is meant the end nearest to the tip or bottom end of the probe. The distal end of the probe is that part of the instrument that is generally embedded in the bony or osseus structure. The distal end of the stimulating wire is the exposed stimulating end of the wire that is exposed at the distal end of the probe.

The probe may be a vertebral distractor, a vertebral post, a screw, a pin or such instruments as may be embedded in bone during surgical procedures such as anterior discectomies and fusions performed upon bony bodies such as anterior vertebral elements. The probe is made of a material capable of penetrating bone which may be stainless steel, ceramic, quartz, titanium or glass, but when the probe is made of a conductive material, the probe may contain a tube housing an insulated stimulating wire.

The stimulating wire may be made of platinum, stainless steel, amidester (Ha), litz, PVC, Teflon®, nylon, gold, silver/silver chloride, silver, titanium, tin or copper, but other suitable materials may also be employed.

In an ideal embodiment, the surgical instrument is connected to a stimulus source capable of providing a stimulus in software controlled intensities, for example by means of a connected processor. In a highly preferred embodiment, the instrument is coupled to a processor capable of synchronizing the stimulus delivered with the recorded motor evoked potentials and monitoring the effects of the stimulus in real-time.

In another preferred embodiment, the stimulating wire proximal end is directly connected to the stimulus source. Alternatively, the wire proximal end further may include a male connector and a female connector to facilitate connection and disconnection.

In one highly preferred embodiment, the probe has a biosensor top or proximal end and the stimulus is provided wirelessly from a remote light source via the biosensor top optically powered by the remote light source. The remote light source is preferably coupled to a computer via a USB port and is infrared. In these types of biosensor embodiments of the surgical instrument, the proximal end of the stimulating wire is connected to the biosensor top end. The biosensor comprises a system-on-a-chip (SOC) attachment including: a stimulus circuit; a receiver means for activating a constant current stimulator to deliver a stimulus via the stimulus circuit; a means for controlling the duration and intensity of the stimulus connected to the stimulus circuit; and a light transmitter/receiver means connected to the SOC attachment and capable of receiving optical power from a remote infra-red transceiver station, and of transmitting a feedback signal thereto. Related patent application Ser. No. 11/292,861 describes a stimulating wireless biosensor electrode, and all that is contained within that application is incorporated by reference.

Also provided are systems comprising one or more of the inventive instruments and at least a processor. In an ideal embodiment, comprises software for providing the stimulus in software controlled intensities. In another highly preferred embodiment, the includes a coupled processor capable of synchronizing the stimulus delivered with the recorded motor evoked potentials and monitoring the effects of the stimulus in real-time, the processor having software for controlled delivery of stimulus, data feedback and real-time monitoring.

The stimulating instruments herein described provide stimulating intensities in the range of between about 0.1 mA and 1000 mA constant current, or between about 0.1 and 400 V constant voltage. Electrical stimulation in train pulse is typically delivered mA and V stimulation in intensities of 100 mA in durations of 0.01 ms to 2.56 ms at rates of 0.1-5 Hz.

The determination of stimulus modalities is based on the amount of resistance met and the level of electrical excitation needed. Constant current stimulation is regarded as ideal for stimulating excitable neural tissue, since resistance tends to increase as stimulation progresses, and a constant current stimulator will sense this increase and adjust to maintain delivery. However, there is a limit to how much voltage a constant current stimulator can provide, therefore when higher levels of electrical excitation were needed, constant voltage stimulator is employed.

The stationary electrical stimulators used are powered by an alternating current (AC) source. Direct constant current in mADC (milli-amperage direct current) and HVPC (high voltage pulsed current) modalities is delivered. Portable electrical stimulators are powered by a dc (direct current) battery source, and are used to deliver mADC (millamperage direct current). The electrical stimulus therefore is either milli-amperage direct current or high voltage pulsed current is delivered, being capable of providing a stimulus of between about 0.10 and 1000 mA, or between about 0.1 and 400 V, respectively.

In one preferred embodiment of the inventive methods, the elicited MMEP and NMEP responses undergo bio-amplification, filtering digital conversion and processing by a data acquisition system, described in detail in related parent application U.S. Ser. No. 11/144,214, which is incorporated by reference. The methodology of the software in U.S. Ser. No. 11/144,214 displays the data, analyzes the data, performs signal conditioning, automated latency amplitude measurements, stores the baseline data then makes real time comparisons of subsequent data and provides audio warning when pathologically significant (predetermined criteria) changes in the data occur.

The recording protocol is as follows:

8 channels

LFF 10 Hz

HFF 3000 Hz

30 ms window uppers/50 ms lowers

| | | | |
|---|---|---|---|
| MMEP: | Left thenar muscle | NMEP: | Left mixed median |
| | Right thenar muscle | | Right mixed median |
| | Left tibialis anterior | | Left mixed posterior tibial |
| | Right tibialis anterior | | Right mixed posterior tibial |

The wirelessly powered biosensor stimulating instruments are powered by a near infrared (ir) light source, an infra red-transceiver station connected via a PC-USB interface to a personal computer. An ir-modulated light beam is directed toward the exposed light collector atop the sensor, the collected light is focused onto a silicon PIN photodiode, and the photodiode converts light into the current needed to operate the sensors electronic components. Power for the biosensor is in the order of microwatts (μW). The architecture of the biosensor consists of variations of data acquisition; data processing; optical communications; power management; I/O expansion; and secondary storage.

The biosensor comprises user-programmable data modulation frequencies, a fast processor and high data throughput. The transceiver station comprises transmitter circuits, controlling a pulsed light emitter, providing a light source that is intensity-modulated to match a light receiver. To produce the highest possible light pulse intensity, a low-duty cycle drive is employed, by driving the LED (complex semiconductors that convert an electrical current into light) with high peak currents with the shortest possible pulse width and with the lowest practical pulse repetition rate. For the sake of efficiency, the LED is driven with a low-loss transistor, and power field effect transistors (FET). Given the long-range application, the LED must be bent into a tight light beam to insure a detectable amount of light reaches the distant receiver. Therefore a wide divergence angle specification is used in calculating lens placement. Multiple light sources or wide area light transmitters may be employed. Angle diversity for non-directed wireless infrared communication, or multi-beam transmitters, with signal splitters, and imaging diversity receiver's principles, may be incorporated in the design.

The infrared LED, a GaAlAs (gallium-aluminum-arsenic) ir-LED, produces light that matches silicon PIN detector response curves. They are packaged in molded plastic assemblies, with small 3/16 lenses. The position of the chip within the package determines the divergence of the exiting light. When used with large lens, it can be used for longer range distances. It also provides receiver circuits which can extract data information that has been placed in the modulated light carrier by the biosensor transmitter and restores the data to its original form. Circuits collect the modulated light from the transmitter with a plastic lens and focus it onto a silicon PIN photodiode, light detectors (PIN)-stray light filters (in reversed biased-mode, it becomes a diode that leaks current in response to light striking it, where the current is directly proportional to the incident light power level, and stray light filters can be placed between the lens and the photodiode), current-to-voltage converter (that converts the current from the PIN to voltage-high impedance detector, resistor feedback, inductor feedback, limited Q), post-signal amplifier (signal filter, noise reduction), signal pulse discriminator (comparator) and decoding circuits (sensor coding, display).

The heart of the sensor is a microprocessor based on an Atmel ATmega 128L™ that operates at 7.372 mHz, and contains 128 kB of on-board flash memory (for storing the program that operates the biosensor) as well as 4 kB EEPROM (for biosensor configuration), 4 kB SRAM (for program memory) and a 16 bit analog. Secondary data storage is handled by an Atmel AT45DB041 serial flash memory array. The 512-kB capacity of this memory array enables the biosensor to locally store or relay over 100,000 measurements to the system's USB port. The infrared transceiver station is able to emit and receive from up to sixteen individual biosensors.

While the instant invention is drawn to different surgical instruments, systems and methods, for delivering a current to an osseus structure such as a vertebral element, the invention should not be limited by the above described embodiments.

What is claimed is:

1. A surgical instrument for eliciting motor evoked potentials intraoperatively, comprising:
an osseus structure probe having a probe distal end, the probe distal end being open; and
a stimulating wire having wire proximal and distal ends, the stimulating wire extending through the probe and being exposed in the open distal end, such that when the probe distal end is embedded in an osseus structure of a subject, and when the stimulating wire proximal end is in contact with a stimulus source, the exposed wire distal end is capable of delivering an electrical stimulus to the surrounding osseus structure.

2. The surgical instrument of claim 1, wherein the stimulating wire distal end is exposed up to 2 mm.

3. The surgical instrument of claim 1, wherein the probe is made of a material capable of penetrating bone.

4. The surgical instrument of claim 1, wherein the probe further comprises an internal tube through which the stimulating wire extends to the probe distal end.

5. The surgical instrument of claim 1, wherein the stimulating wire is coated with an electrical insulation material.

6. The surgical instrument of claim 1, wherein the stimulating wire is made of a substance selected from the group consisting of platinum, stainless steel, amidester (Ha), litz, PVC, Teflon®, nylon, gold, silver/silver chloride, silver, titanium, tin and copper.

7. The surgical instrument of claim 1, wherein the osseus structure is a vertebral element.

8. The surgical instrument of claim 1, wherein the stimulus is provided in software controlled intensities.

9. The surgical instrument of claim 1, coupled to a computer monitoring the effects of the stimulus in real-time.

10. The surgical instrument of claim 1, wherein the electrical stimulus is milli-amperage direct current.

11. The surgical instrument of claim 10, wherein between 0.10 and 1,000 mA is delivered.

12. The surgical instrument of claim 1, wherein high voltage pulsed current is delivered.

13. The surgical instrument of claim 12, wherein between 0.1 and 400 V is delivered.

14. The surgical instrument of claim 1, wherein the wire proximal end is connected to the stimulus source.

15. The surgical instrument of claim 14, wherein the wire proximal end further comprises a male connector and a female connector.

16. The surgical instrument of claim 1, wherein the stimulus is provided wirelessly from a remote light source.

17. The surgical instrument claim 16, wherein the remote light source is coupled to a computer via a USB port.

18. The surgical instrument of claim 16, wherein the light source is infrared.

19. The surgical instrument of claim 16, wherein the instrument further comprises a biosensor top end, wherein the proximal end is connected to the biosensor top end, and wherein the biosensor top is optically powered by the remote light source.

20. The surgical instrument of claim 19, wherein the biosensor top comprises a system-on-a-chip (SOC) attachment including: a stimulus circuit; a receiver means for activating a constant current stimulator to deliver a stimulus via the stimulus circuit; a means for controlling the duration and intensity of the stimulus connected to the stimulus circuit; and a light transmitter/receiver means connected to the SOC attachment and capable of receiving optical power from the remote transceiver, and of transmitting a feedback signal thereto.

21. The surgical instrument of claim 1, wherein the probe is one of: a vertebral distractor, a vertebral post, a screw and a pin.

22. The surgical instrument of claim 1, wherein the probe is made of stainless steel, ceramic, quartz, titanium or glass.

23. A system comprising the instrument of one or more of the instrument of claim 1, and at least a processor.

24. The system of claim 23, further comprising software for controlled delivery of stimulus, data feedback and real-time monitoring.

25. An method of eliciting an evoked potential in a subject during a surgical procedure being performed on the subject by delivering a stimulus to the osseus structure of the subject, the method comprising the steps of:
connecting to a subject's osseus structure a surgical instrument, comprising: an osseus structure probe; and a stimulating wire extending through the probe to, and being exposed at its distal end at, the open probe distal end;
putting the stimulating wire proximal end in connection with a stimulus source and delivering a stimulus to the osseus structure via the exposed wire distal end; and
measuring an evoked potential elicited from the subject as a result of the delivered stimulus.

26. The method of claim 25, wherein the osseus structure is a vertebral element.

27. The method of claim 25, wherein the instrument is embedded in the osseus structure.

28. The method of claim 25, wherein the probe is one of: a vertebral distractor, a vertebral post, a screw, or a pin.

29. The method of claim 25, further comprising providing the stimulus in software controlled intensities.

30. The method of claim 25, further comprising monitoring the effects of the stimulus in real-time.

31. The method of claim 25, wherein the stimulus is milli-amperage direct current.

32. The method of claim 31, wherein between 0.10 and 1,000 mA is delivered.

33. The method of claim 25, wherein the stimulus is high voltage pulsed current.

34. The method of claim 33, wherein between 0.1 and 400 V is delivered.

35. The method of claim 25, wherein the wire proximal end is directly connected to a stimulus source.

36. The method of claim 25, wherein the wire proximal end is wirelessly powered by a remo light source.

37. The method of claim 36, wherein the remote light source is coupled to a computer via a USB port.

38. The method of claim 36, wherein the light source is infrared.
